(19) United States
(12) Patent Application Publication
(10) Pub. No.: US 2024/0226507 A1
(43) Pub. Date: Jul. 11, 2024

(54) Catheter Insertion Assemblies, Introducer-Needle Subassemblies, and Methods with Needle-Stick-Injury Protection

(71) Applicant: Bard Access Systems, Inc., Salt Lake City, UT (US)

(72) Inventor: Joe Spataro, Cottonwood Heights, UT (US)

(21) Appl. No.: 18/410,781

(22) Filed: Jan. 11, 2024

Related U.S. Application Data
(60) Provisional application No. 63/438,474, filed on Jan. 11, 2023.

(51) Int. Cl. A61M 25/09; A61M 25/00; A61M 25/06

(52) U.S. Cl. CPC A61M 25/09; A61M 25/0026; A61M 25/0606

(57) ABSTRACT

A RICC insertion assembly can include a RICC, an introducer needle, and a coupler coupling the RICC and the introducer needle together. The introducer needle can include a needle hub over a proximal portion of a composite shaft including a sheath over a needle shaft sealing a needle slot of the needle shaft thereunder. The coupler can include a coupler housing and a valve module disposed in the coupler housing. The composite shaft can pass through an introducer-needle passageway defined by a valve-module housing of the valve module such that the valve module forms a seal over a sheath opening of the sheath in a ready-to-deploy state of the RICC insertion assembly. The valve module can include a needle-stick-injury protection mechanism configured to capture a distal needle tip of the needle shaft within the valve module when the introducer needle is withdrawn from the coupler in a proximal direction.

### PRIORITY

This application claims the benefit of priority to U.S. Provisional Patent Application No. 63/438,474, filed Jan. 11, 2023, which is incorporated by reference in its entirety into this application.

### BACKGROUND

Central venous catheter (“CVCs”) are commonly introduced into patients and advanced through their vasculatures for placement by way of the Seldinger technique. The Seldinger technique utilizes a number of steps and medical devices (e.g., a needle, a scalpel, a guidewire, an introducer sheath, a dilator, a CVC, etc.). While the Seldinger technique is effective, the number of steps are time consuming, handling the number of medical devices is awkward, and both of the foregoing can lead to patient trauma. In addition, there is a relatively high potential for touch contamination due to the number of medical devices that need to be interchanged during the Seldinger technique. As such, there is a need to reduce the number of steps and medical devices involved in introducing a catheter such as a CVC into a patient and advancing the catheter through a vasculature thereof.

Disclosed herein are rapidly insertable central catheter (“RICC”) insertion assemblies, introducer-needle assemblies, and methods that address the foregoing. Notably such RICC insertion assemblies, introducer-needle assemblies, and methods include needle-stick-injury protection, as set forth below.

### SUMMARY

Disclosed herein is a RICC insertion assembly including, in some embodiments, a RICC, an introducer needle, and a coupler coupling the RICC and the introducer needle together. The introducer needle includes a needle hub over a proximal portion of a composite shaft. The composite shaft includes a needle shaft having a longitudinal needle slot and a sheath over the needle shaft sealing the needle slot thereunder but for a sheath opening in the sheath to the needle slot. The coupler includes a coupler housing and a valve module disposed in the coupler housing. The composite shaft passes through an introducer-needle passageway defined by a valve-module housing of the valve module such that the valve module forms a seal over the sheath opening in a ready-to-deploy state of the RICC insertion assembly. The valve module includes a needle-stick-injury (“NSI”) protection mechanism configured to capture a distal needle tip of the needle shaft within the valve module when the introducer needle is withdrawn from the coupler in a proximal direction.

In some embodiments, the NSI protection mechanism includes a tether having a proximal portion coupled to the needle hub and a distal portion coupled to the valve-module housing.

In some embodiments, the tether has a fixed tether length configured to prevent the needle tip of the needle shaft from escaping the valve module when the introducer needle is withdrawn from the coupler in the proximal direction or thereafter.

In some embodiments, the tether includes a plurality of pleats and a plurality of pleat through holes through the pleats. The composite shaft is disposed in the pleat through holes such that the pleats unfold over the composite shaft when the introducer needle is withdrawn from the coupler in a proximal direction.

In some embodiments, the NSI protection mechanism includes a spring loaded in a spring cavity along a portion of the introducer-needle passageway. The spring is configured to unload into the introducer-needle passageway when the introducer needle is withdrawn from the coupler in the proximal direction and the needle tip of the needle shaft passes the spring.

In some embodiments, the spring is a flat spring including an end tab configured to extend across the introducer-needle passageway into a tab receiver of the valve-module housing opposite the spring cavity at the time the needle tip of the needle shaft passes the spring. This prevents the needle tip of the needle shaft from escaping the valve module if the introducer needle is advanced into the coupler in a distal direction.

In some embodiments, the end tab of the spring provides a primary tip shield, and a fulcrum of the spring provides a secondary tip shield if the needle tip of the needle shaft is forcibly advanced into the coupler in the distal direction past the end tab.

In some embodiments, the fulcrum of the spring is between a free arm of the spring including the end tab and a fixed arm of the spring fixed to the valve-module housing in the spring cavity.

In some embodiments, the RICC insertion assembly further includes an access guidewire. The access guidewire includes a distal portion having a distal end. The distal portion of the access guidewire passes through a guidewire passageway defined by the valve-module housing of the valve module, a continuation of the guidewire passageway defined by an elastomeric gasket of the valve module, the sheath opening in the sheath, and an introducer-needle lumen of the introducer needle such that the distal end of the access guidewire is disposed just proximal of the needle tip of the needle shaft in the ready-to-deploy state of the RICC insertion assembly.

In some embodiments, the valve module includes a blade extending into the sheath opening of the sheath from the valve-module housing to which the blade is fixed. The blade is configured to cut the sheath along the needle slot when the introducer needle is withdrawn from the coupler in a proximal direction. This allows the access guidewire to escape from the introducer-needle lumen of the introducer needle.

In some embodiments, the RICC includes a catheter tip, a catheter tube, a catheter hub, and one or more extension legs. The catheter tip is coupled to a distal end portion of the catheter tube. The catheter tube includes a catheter-tube portion of a primary lumen of the RICC. The catheter hub is coupled to a proximal portion of the catheter tube. Each extension leg of the one-or-more extension legs is coupled to the catheter hub by a distal portion thereof.

In some embodiments, the catheter tip is of a first polymeric material and the catheter tube is of a second, softer polymeric material.

In some embodiments, the first polymeric material is sufficiently stiff to prevent the catheter tip from collapsing, buckling, or otherwise appreciably deforming when a distal portion of the RICC is advanced into a blood-vessel lumen of a patient. The first polymeric material is also sufficiently pliable to prevent trauma to the blood-vessel lumen when the distal portion of the RICC is advanced farther into the blood-vessel lumen.

In some embodiments, the RICC includes a set of three lumens including the primary lumen, a secondary lumen, and a tertiary lumen formed of fluidly connected portions of at least three catheter-tube lumens, three catheter-hub lumens, and three extension-leg lumens. The secondary and tertiary lumens terminate in the distal end portion of the catheter tube by at least some infill of melted polymeric material of the first polymeric material.

In some embodiments, the primary lumen has a primary-lumen aperture in a distal end of the catheter tip, the secondary lumen has a secondary-lumen aperture in a side of the distal portion of the catheter tube, and the tertiary lumen has a tertiary-lumen aperture in the side of the distal end portion of the catheter tube but proximal of the secondary-lumen aperture.

In some embodiments, the RICC insertion assembly of further includes a syringe. The syringe is fluidly coupled to the needle hub of the introducer needle in at least the ready-to-deploy state of the RICC insertion assembly.

Also disclosed herein is an introducer-needle subassembly including, in some embodiments, an introducer needle and a coupler coupled to the introducer needle. The introducer needle includes a needle hub over a proximal portion of a composite shaft. The composite shaft includes a needle shaft having a longitudinal needle slot and a sheath over the needle shaft sealing the needle slot thereunder but for a sheath opening in the sheath to the needle slot. The coupler includes a coupler housing and a valve module disposed in the coupler housing. The composite shaft passes through an introducer-needle passageway defined by a valve-module housing of the valve module such that the valve module forms a seal over the sheath opening in a ready-to-deploy state of the introducer-needle subassembly. The valve module includes an NSI protection mechanism configured to capture a distal needle tip of the needle shaft within the valve module when the introducer needle is withdrawn from the coupler in a proximal direction.

In some embodiments, the NSI protection mechanism includes a tether having a proximal portion coupled to the needle hub and a distal portion coupled to the valve-module housing.

In some embodiments, the tether has a fixed tether length configured to prevent the needle tip of the needle shaft from escaping the valve module when the introducer needle is withdrawn from the coupler in the proximal direction or thereafter.

In some embodiments, the tether includes a plurality of pleats and a plurality of pleat through holes through the pleats. The composite shaft is disposed in the pleat through holes such that the pleats unfold over the composite shaft when the introducer needle is withdrawn from the coupler in a proximal direction.

In some embodiments, the NSI protection mechanism includes a spring loaded in a spring cavity along a portion of the introducer-needle passageway. The spring is configured to unload into the introducer-needle passageway when the introducer needle is withdrawn from the coupler in the proximal direction and the needle tip of the needle shaft passes the spring.

In some embodiments, the spring is a flat spring including an end tab configured to extend across the introducer-needle passageway into a tab receiver of the valve-module housing opposite the spring cavity at the time the needle tip of the needle shaft passes the spring. This prevents the needle tip of the needle shaft from escaping the valve module if the introducer needle is advanced into the coupler in a distal direction.

In some embodiments, the end tab of the spring provides a primary tip shield, and a fulcrum of the spring provides a secondary tip shield if the needle tip of the needle shaft is forcibly advanced into the coupler in the distal direction past the end tab.

In some embodiments, the fulcrum of the spring is between a free arm of the spring including the end tab and a fixed arm of the spring fixed to the valve-module housing in the spring cavity.

Also disclosed herein is a method of an introducer-needle subassembly. The method includes, in some embodiments, a needle tract-establishing step, an access guidewire-advancing step, an introducer needle-withdrawing step, and a needle tip-capturing step. The needle tract-establishing step includes establishing a needle tract from a skin surface to a blood vessel of a patient with an introducer needle of the introducer-needle subassembly. The introducer needle includes a needle hub over a proximal portion of a composite shaft. The composite shaft includes a needle shaft having a longitudinal needle slot and a sheath over the needle shaft sealing the needle slot thereunder but for a sheath opening in the sheath to the needle slot. The access guidewire-advancing step includes advancing a distal end of an access guidewire from just proximal of a distal needle tip of the needle shaft into the blood vessel. The introducer needle-withdrawing step includes withdrawing the introducer needle in a proximal direction from the needle tract leaving the access guidewire in place. The introducer needle-withdrawing step also includes further withdrawing the introducer needle in the proximal direction from a coupler coupling the introducer needle and the access guidewire together. The coupler includes a coupler housing and a valve module having a valve-module housing is disposed in the coupler housing. The needle tip-capturing step includes capturing the needle tip of the needle shaft within the valve module by way of an NSI protection mechanism.

In some embodiments, the NSI protection mechanism includes a tether having a proximal portion coupled to the needle hub and a distal portion coupled to the valve-module housing.

In some embodiments, the tether has a fixed tether length configured to prevent the needle tip of the needle shaft from escaping the valve module during the further withdrawing of the introducer needle from the coupler in the introducer needle-withdrawing step or thereafter.

In some embodiments, the tether includes a plurality of pleats and a plurality of pleat through holes through the pleats. The composite shaft is disposed in the pleat through holes such that the pleats unfold over the composite shaft during the further withdrawing of the introducer needle from the coupler in the introducer needle-withdrawing step.

In some embodiments, the NSI protection mechanism includes a spring loaded in a spring cavity along a portion of an introducer-needle passageway defined by the valve-module housing of the valve module. The spring unloads into the introducer-needle passageway during the further withdrawing of the introducer needle from the coupler in the introducer needle-withdrawing step at a time when the needle tip of the needle shaft passes the spring.

In some embodiments, the spring is a flat spring including an end tab configured to extend across the introducer-needle passageway into a tab receiver of the valve-module housing opposite the spring cavity at the time the needle tip of the needle shaft passes the spring. This prevents the needle tip of the needle shaft from escaping the valve module if the introducer needle is advanced into the coupler in a distal direction.

In some embodiments, the end tab of the spring provides a primary tip shield, and a fulcrum of the spring provides a secondary tip shield if the needle tip of the needle shaft is forcibly advanced into the coupler in the distal direction past the end tab.

In some embodiments, the fulcrum of the spring is between a free arm of the spring including the end tab and a fixed arm of the spring fixed to the valve-module housing in the spring cavity.

These and other features of the concepts provided herein will become more apparent to those of skill in the art in view of the accompanying drawings and following description, which describe particular embodiments of such concepts in greater detail.

### BRIEF DESCRIPTIONS OF DRAWINGS

FIG. 1 illustrates a RICC insertion assembly in accordance with some embodiments.

FIG. 2 illustrates an introducer-needle subassembly of the RICC insertion assembly in accordance with some embodiments.

FIG. 3 illustrates a longitudinal cross section of the introducer-needle subassembly with a spring of an NSI protection mechanism loaded in a spring cavity in accordance with some embodiments.

FIG. 4 illustrates a longitudinal cross section of the introducer-needle subassembly with the spring of the NSI protection mechanism unloaded from the spring cavity in accordance with some embodiments.

FIG. 5 illustrates a detailed view of the longitudinal cross section of the introducer-needle subassembly of FIG. 4 in accordance with some embodiments.

FIG. 6 illustrates a detailed isometric view of the introducer-needle subassembly with the spring of the NSI protection mechanism unloaded from the spring cavity in accordance with some embodiments.

FIG. 7 illustrates a composite shaft of an introducer needle including a needle shaft and a sheath over the needle shaft in accordance with some embodiments.

FIG. 8 illustrates the sheath in accordance with some embodiments.

FIG. 9 illustrates the needle shaft in accordance with some embodiments.

FIG. 10 illustrates a RICC in accordance with some embodiments.

FIG. 11 illustrates a distal end portion of a catheter tube of the RICC in accordance with some embodiments.

FIG. 12 illustrates a longitudinal cross section of the distal end portion of the catheter tube in accordance with some embodiments.

FIG. 13 illustrates a transverse cross section of a catheter tip coupled to the catheter tube in accordance with some embodiments.

FIG. 14 illustrates another transverse cross section of the catheter tip in accordance with some embodiments.

FIG. 15 illustrates a transverse cross section of the distal end portion of the catheter tube in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

“Proximal” is used to indicate a portion, section, piece, element, or the like of a medical device intended to be near or relatively nearer to a clinician when the medical device is used on a patient. For example, a “proximal portion” or “proximal section” of the medical device includes a portion or section of the medical device intended to be near the clinician when the medical device is used on the patient. Likewise, a “proximal length” of the medical device includes a length of the medical device intended to be near the clinician when the medical device is used on the patient. A “proximal end” of the medical device is an end of the medical device intended to be near the clinician when the medical device is used on the patient. The proximal portion, the proximal section, or the proximal length of the medical device need not include the proximal end of the medical device. Indeed, the proximal portion, the proximal section, or the proximal length of the medical device can be short of the proximal end of the medical device. However, the proximal portion, the proximal section, or the proximal length of the medical device can include the proximal end of the medical device. Should context not suggest the proximal portion, the proximal section, or the proximal length of the medical device includes the proximal end of the medical device, or if it is deemed expedient in the following description, “proximal portion,” “proximal section,” or “proximal length” can be modified to indicate such a portion, section, or length includes an end portion, an end section, or an end length of the medical device for a “proximal end portion,” a “proximal end section,” or a “proximal end length” of the medical device, respectively.

“Distal” is used to indicate a portion, section, piece, element, or the like of a medical device intended to be near, relatively nearer, or even in a patient when the medical device is used on the patient. For example, a “distal portion” or “distal section” of the medical device includes a portion or section of the medical device intended to be near, relatively nearer, or even in the patient when the medical device is used on the patient. Likewise, a “distal length” of the medical device includes a length of the medical device intended to be near, relatively nearer, or even in the patient when the medical device is used on the patient. A “distal end” of the medical device is an end of the medical device intended to be near, relatively nearer, or even in the patient when the medical device is used on the patient. The distal portion, the distal section, or the distal length of the medical device need not include the distal end of the medical device. Indeed, the distal portion, the distal section, or the distal length of the medical device can be short of the distal end of the medical device. However, the distal portion, the distal section, or the distal length of the medical device can include the distal end of the medical device. Should context not suggest the distal portion, the distal section, or the distal length of the medical device includes the distal end of the medical device, or if it is deemed expedient in the following description, “distal portion,” “distal section,” or “distal length” can be modified to indicate such a portion, section, or length includes an end portion, an end section, or an end length of the medical device for a “distal end portion,” a “distal end section,” or a “distal end length” of the medical device, respectively.

Unless defined otherwise, all technical and scientific terms used herein have the same meaning as commonly understood by those of ordinary skill in the art.

Again, disclosed herein are RICC insertion assemblies, introducer-needle assemblies, and methods, particularly those including needle-stick-injury protection, as set forth below. Such RICC insertion assemblies, introducer-needle assemblies, and methods will become more apparent to those of skill in the art in view of the accompanying drawings and following description, which describe particular embodiments of the foregoing mostly in the context of RICC insertion assemblies. But the RICC insertion assemblies include but one type of catheter that can be incorporated into catheter insertion assemblies like the RICC insertion assemblies provided herein. Indeed, peripherally inserted central catheters (“PICCs”), dialysis catheters, or the like can also be incorporated into catheter insertion assemblies for rapid insertion or otherwise.

### RICC Insertion Assemblies

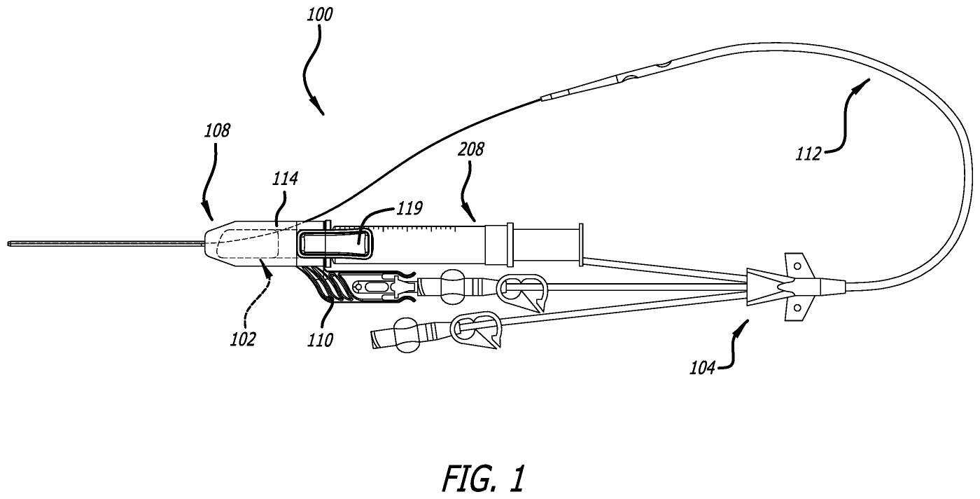

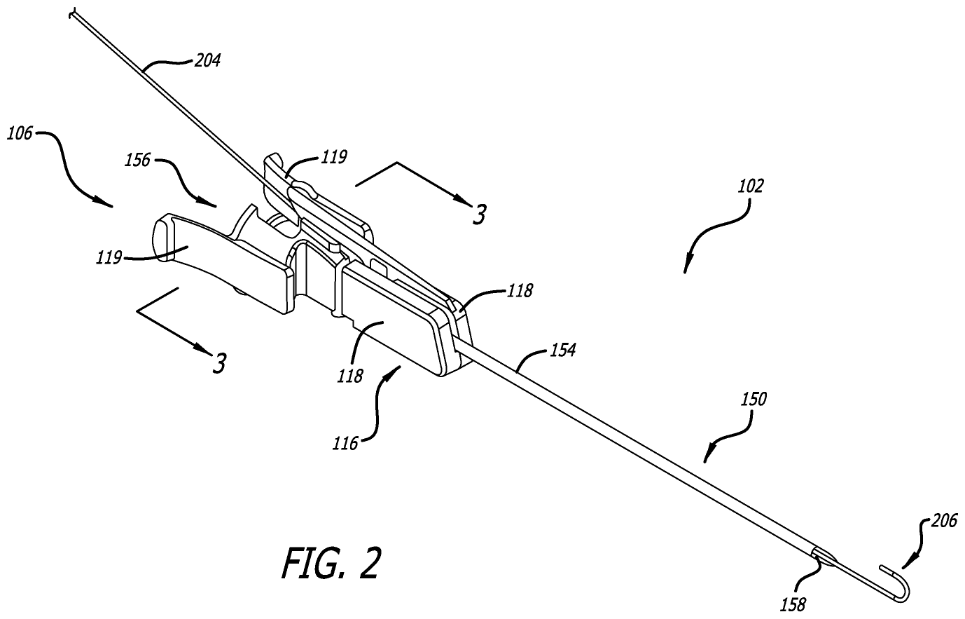

FIG. 1 illustrates a RICC insertion assembly 100 in accordance with some embodiments. FIG. 2 illustrates an introducer-needle subassembly 102 of the RICC insertion assembly 100 in accordance with some embodiments.

As shown, the RICC insertion assembly 100 can include a RICC 104, an introducer needle 106, and a coupler 108 coupling the RICC 104 and the introducer needle 106 together. Notably, the introducer-needle subassembly 102 can include the introducer needle 106 and the coupler 108 coupled to the introducer needle 106. As set forth below for the RICC insertion assembly 100, the introducer-needle subassembly 102 can likewise further include the access guidewire 204 in accordance with some embodiments. In at least a ready-to-deploy state of the RICC insertion assembly 100, the proximal end of the access guidewire 204 can be attached to an extension arm 110 of the coupler 108 or held in location by some other holding means for holding the access guidewire 204 including by hand, and the distal end of the access guidewire 204 can be disposed in the needle lumen 164 of the introducer needle 106 as set forth below. This can enforce a loop 112 in the access guidewire 204. The RICC 104 can be disposed over the loop 112 in the ready-to-deploy state of the RICC insertion assembly 100, thereby keeping the RICC insertion assembly 100 in a relatively compact form.

As shown, the coupler 108 can include a coupler housing 114 and a valve module 116 having a valve-module housing 118 disposed in the coupler housing 114. While shown as such, it should be understood the valve-module housing 118 can be integrated with the coupler housing 114 in an integrated housing in some embodiments.

The coupler housing 114 can include two molded pieces coupled together (e.g., coupled together with compression pins or heat-staked pins, fastened or screwed together with screws or bolts, welded together with an ultrasonic weld or a hot-plate weld, etc.) to form a body, as shown, which body can be configured to be comfortably held underhand (e.g., cradled) in either a left hand for a left-handed venipuncture or a right hand for a right-handed venipuncture with the introducer needle 106 of the RICC insertion assembly 100. While not shown, an inside of each piece of the two molded pieces can include a distal depression that forms a valve-module compartment for the valve module 116 when the two molded pieces are coupled together. Alternatively, the distal depression in each piece of the two molded pieces can similarly form the gasket compartment, which gasket compartment can be configured for the elastomeric gasket 120 set forth below in the embodiments of the integrated housing set forth above. In either case, the inside of each piece of the two molded pieces can further include a proximal depression that forms a needle-hub receptacle for the needle hub 156 of the introducer needle 106 when the two molded pieces are coupled together. As evidenced by clip arms 119 of the needle hub 156, the coupler housing 114 can further include tabs outboard of the needle-hub receptacle for clipping the clip arms 119 thereover. Lastly, the coupler housing 114 can include a longitudinal coupler-housing slot between the two molded pieces when the two molded pieces are coupled together. The coupler-housing slot can open in a same or different direction as the needle slot 160 of the needle shaft 152, thereby configuring the coupler-housing slot to allow the access guidewire 204 to escape from the coupler housing 114 after the introducer needle 106 is withdrawn from the coupler 108 in the proximal direction in accordance with the introducer needle-withdrawing step of the method set forth below.

Like the coupler housing 114, the valve-module housing 118 can include two molded pieces coupled together in the valve-module compartment to form a gasket compartment for an elastomeric gasket 120. The valve-module compartment of the coupler housing 114 can be configured with sufficient space to allow the valve-module housing 118 and the elastomeric gasket 120 disposed therein to separate for the escape of the access guidewire 204 from the valve module 116 after the introducer needle 106 is withdrawn from the coupler 108 in the proximal direction in accordance with the introducer needle-withdrawing step of the method set forth below. Notably, the introducer needle 106 is withdrawn in the proximal direction through an introducer-needle passageway 122 in which the composite shaft 150 of the introducer needle 106 is disposed in at least the ready-to-deploy state of the RICC insertion assembly 100. The introducer-needle passageway 122 can be defined by a combination of coupler components selected from the coupler housing 114, the valve-module housing 118, and the elastomeric gasket 120. Likewise, a guidewire passageway 124 connected to the introducer-needle passageway 122 in the valve module 116 can be defined by the combination of coupler components selected from the coupler housing 114, the valve-module housing 118, and the elastomeric gasket 120. By way of the guidewire passageway 124 connecting with the introducer-needle passageway 122 in the valve module 116, as well as the sheath opening 168 of the sheath 154 to the needle slot 160 of the needle shaft 152 set forth below, the access guidewire 204 can be disposed in the composite shaft 150 of the introducer needle 106 while the composite shaft 150 of the introducer needle 106 is disposed in the introducer-needle passageway 122.

The coupler 108 can further include a blade 126 for cutting the sheath 154 along the needle slot 160 of the needle shaft 152. For example, the valve module 116 can include the blade 126 distal of the spring 128 that extends into the sheath opening 168 of the sheath 154 from the valve-module housing 118 to which the blade 126 is fixed. The blade 126 can be configured to cut the sheath 154 along the needle slot 160 when the introducer needle 106 is withdrawn from the coupler 108 in the proximal direction in accordance with the introducer needle-withdrawing step of the method set forth below, which allows the access guidewire 204 to escape from the needle lumen 164 of the introducer needle 106.

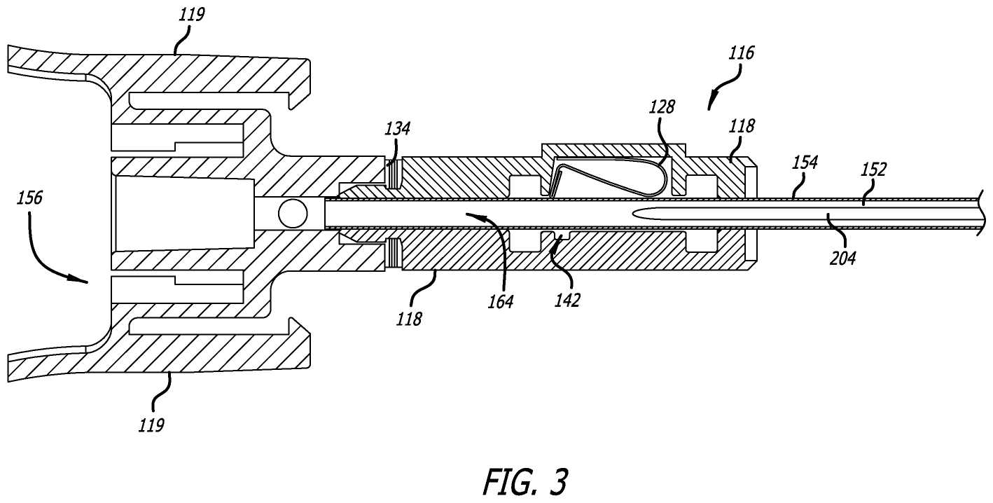

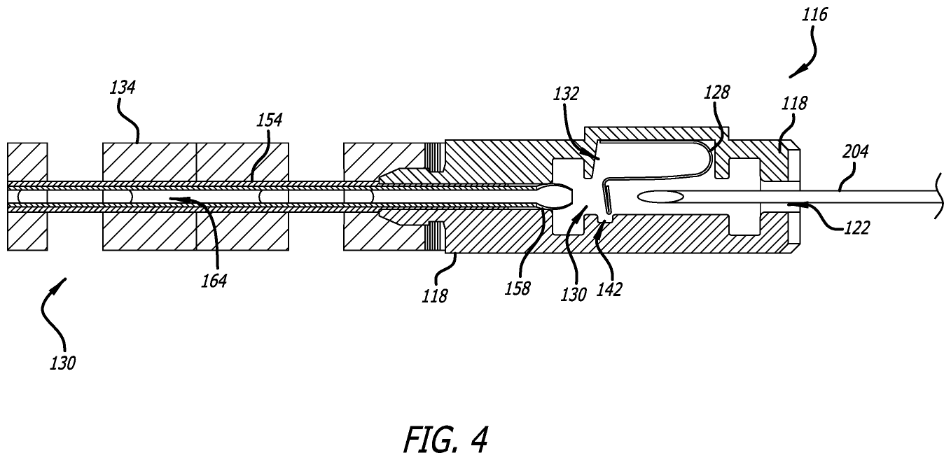

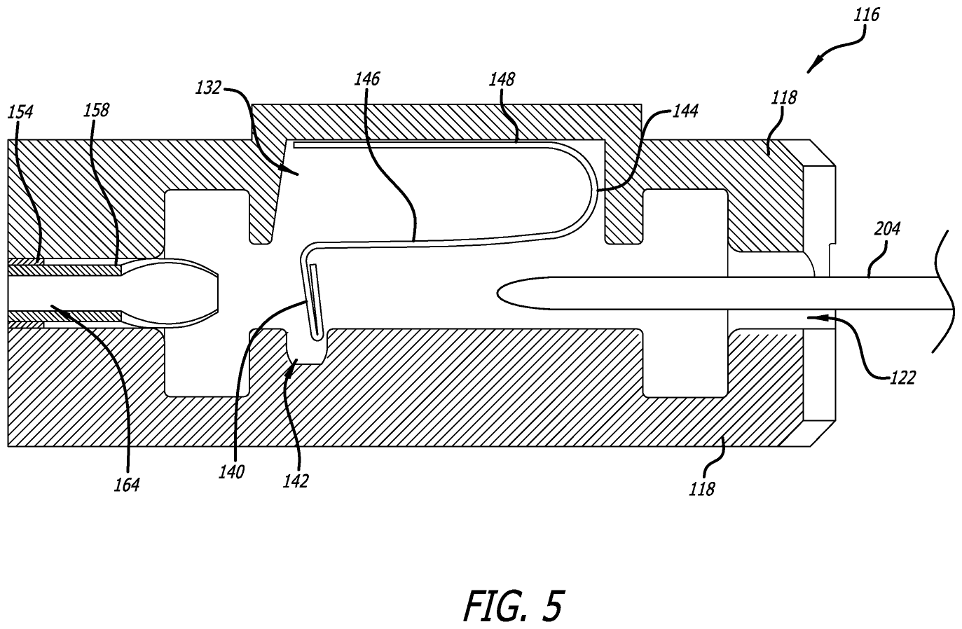

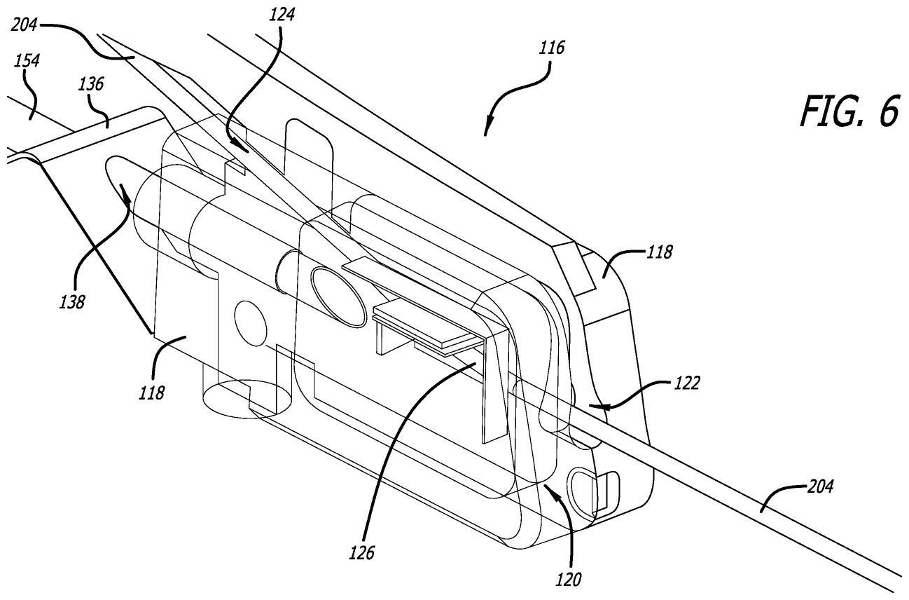

FIG. 3 illustrates a longitudinal cross section of the introducer-needle subassembly 102 with a spring 128 of an NSI protection mechanism 130 loaded in a spring cavity 132 in accordance with some embodiments. FIGS. 4-6 illustrate various views of the introducer-needle subassembly 102 with the spring 128 of the NSI protection mechanism 130 unloaded from the spring cavity 132 in accordance with some embodiments.

As shown, the coupler 108 can further include the NSI protection mechanism 130 configured to capture the needle tip 158 of the needle shaft 152 subsequent to establishing a needle tract in accordance with the needle tract-establishing step of the method set forth below. For example, the valve module 116 can include the NSI protection mechanism 130 configured to capture the needle tip 158 of the needle shaft 152 within the valve module 116 when the introducer needle 106 is withdrawn from the coupler 108 in the proximal direction in accordance with the introducer needle-withdrawing step of the method set forth below.

The NSI protection mechanism 130 can include a tether 134 having a proximal portion coupled to the needle hub 156 and a distal portion coupled to the valve-module housing 118 or the coupler housing 114 in some embodiments. The tether 134 can have a fixed tether length configured to prevent the needle tip 158 of the needle shaft 152 from escaping the valve module 116 when the introducer needle 106 is withdrawn from the coupler 108 in the proximal direction in accordance with the introducer needle-withdrawing step of the method set forth below or thereafter. Notably, the tether 134 can include a plurality of pleats 136 and a plurality of pleat through holes 138 through the pleats 136. The composite shaft 150 of the introducer needle 106 can be disposed in the pleat through holes 138 such that the pleats 136 unfold over the composite shaft 150 when the introducer needle 106 is withdrawn from the coupler 108 in the proximal direction in accordance with the introducer needle-withdrawing step of the method set forth below.

The NSI protection mechanism 130 can further include the spring 128 loaded in the spring cavity 132 along a portion of the introducer-needle passageway 122. Such a spring cavity 132 can be defined by the valve-module housing 118; however, the NSI protection mechanism 130 can be extended outside of the valve module 116 such that the spring cavity 132 is defined by the coupler housing 114. Regardless, the spring 128 can be configured to unload into the introducer-needle passageway 122 when the introducer needle 106 is withdrawn from the coupler 108 in the proximal direction in accordance with the introducer needle-withdrawing step of the method set forth below and the needle tip 158 of the needle shaft 152 passes the spring 128.

The spring 128 of the NSI protection mechanism 130 can be a flat spring including an end tab 140 dimensioned or otherwise configured to extend across the introducer-needle passageway 122 into a tab receiver 142 of the valve-module housing 118 or coupler housing 114 opposite the spring cavity 132 at the time the needle tip 158 of the needle shaft 152 passes the spring 128. Such a tab receiver 142 can be another, smaller cavity along the foregoing portion of the introducer-needle passageway 122 opposite the spring cavity 132. Once in place in the tab receiver 142, the end tab 140 can prevent the needle tip 158 of the needle shaft 152 from escaping the coupler 108 or the valve module 116 thereof if the introducer needle 106 is advanced into the coupler 108 in a distal direction. Notably, the end tab 140 of the spring 128 can provide a primary tip shield and a fulcrum 144 of the spring 128 can provide a secondary tip shield if the needle tip 158 of the needle shaft 152 is forcibly advanced into the coupler 108 in the distal direction past the end tab 140. As shown, the fulcrum 144 of the spring 128 is between a free arm 146 of the spring 128 including the end tab 140 and a fixed arm 148 of the spring 128 fixed to the valve-module housing 118 or the coupler housing 114 in the spring cavity 132.

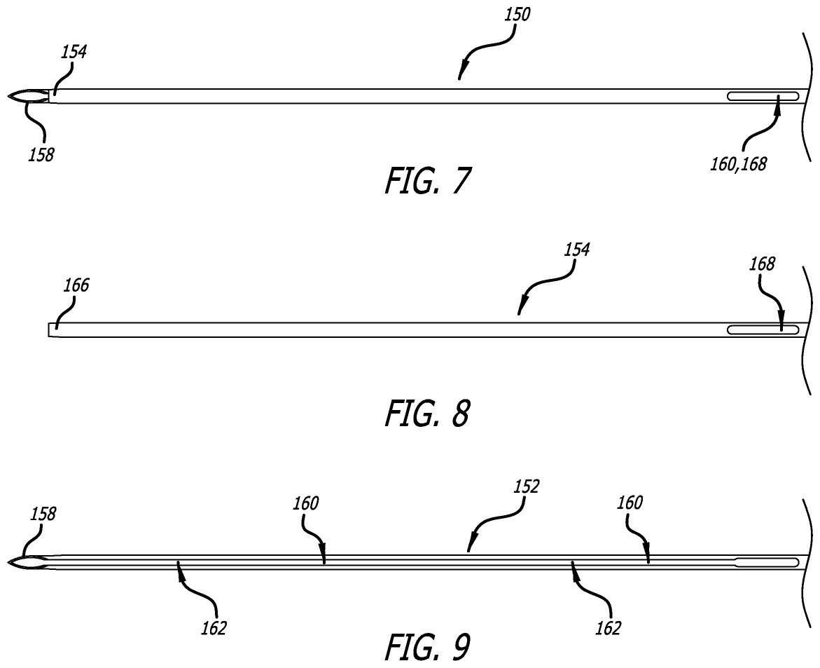

FIG. 7 illustrates a composite shaft 150 of the introducer needle 106 including a needle shaft 152 and a sheath 154 over the needle shaft 152 in accordance with some embodiments. FIG. 8 illustrates the sheath 154 in accordance with some embodiments, while FIG. 9 illustrates the needle shaft 152 in accordance with some embodiments.

The introducer needle 106 can include a needle hub 156 over a proximal portion of the composite shaft 150. In at least the ready-to-deploy state of the RICC insertion assembly 100, the composite shaft 150 can pass through the introducer-needle passageway 122 including that portion of the introducer-needle passageway 122 through the valve module 116, which, in turn, includes the valve-module housing 118 and gasket thereof, such that the valve module 116 forms a seal over the sheath opening 168.

The composite shaft 150 can include the needle shaft 152 and the sheath 154 over the needle shaft 152 sealing the needle slot 160 thereunder but for the sheath opening 168 in the sheath 154 to the needle slot 160, as set forth below.

The needle shaft 152 can include a distal needle tip 158 in a distal portion of the needle shaft 152 and a longitudinal needle slot 160 extending from a proximal portion of the needle shaft 152 through the needle tip 158. The needle slot 160 forms a needle channel 162 along a majority of a length of the needle shaft 152 as opposed to the needle lumen 164 therethrough, as set forth below. The needle slot 160 can have a width sized in accordance with an outer diameter of the access guidewire 204, which allows the access guidewire 204 to pass from the proximal portion of the needle shaft 152 through the needle tip 158 in accordance with the introducer needle-withdrawing step of the method set forth below.

While the needle shaft 152 can include the needle slot 160, it should be understood the introducer needle 106 or the composite shaft 150 thereof can include a needle lumen 164. Indeed, the needle lumen 164 results from a combination of the needle shaft 152 and the sheath 154 over the needle shaft 152. That is, the sheath 154 over the needle shaft 152 can seal the needle slot 160 thereunder forming the needle lumen 164 of the introducer needle 106 or the composite shaft 150 thereof, thereby enabling the syringe 208 to aspirate blood, for example, to confirm blood-vessel access after establishing the needle tract in accordance with the needle tract-establishing step of the method set forth below.

The sheath 154 can include a sheath tip 166 in a distal portion of the sheath 154 and a sheath opening 168 in a side of the proximal portion of the sheath 154.

The sheath tip 166 can include a relatively short taper from an outer diameter of the distal portion of the sheath 154 to an outer diameter of a distal end of the sheath 154, the latter of which can be commensurate with an outer diameter of the distal portion of the needle shaft 152.

The sheath opening 168 can open to the needle slot 160 of the needle shaft 152 allowing the access guidewire 204 to pass through the sheath opening 168 and into the needle channel 162 or the needle lumen 164 in at least the ready-to-deploy state of the RICC insertion assembly 100. Thus, the sheath opening 168 can have a width approximately commensurate with a width of the needle slot 160, which, in turn, can be sized in accordance with the diameter of the access guidewire 204. The sheath opening 168 can also have a length sufficient to allow the access guidewire 204 to pass through the sheath opening 168 and into the needle slot 160 or the needle lumen 164 while also accommodating the blade 126 of the valve module 116 under a distal end of the sheath opening 168. Notably, the sheath 154 over the needle shaft 152 can seal the needle slot 160 thereunder except for that portion of the needle slot 160 under the sheath opening 168. However, the valve module 116 can seal over the needle slot 160 exposed by the sheath opening 168 by sealing the proximal portions of the needle shaft 152 and the sheath 154 therein, thereby enabling the syringe 208 to aspirate blood, for example, to confirm blood-vessel access after establishing the needle tract in accordance with the needle tract-establishing step of the method set forth below.

The sheath 154, or a sheath body thereof, can be formed of a polymeric material configured to facilitate a smooth, consistent insertion of the introducer needle 106 from an area of skin to a blood-vessel lumen of a patient in accordance with the needle tract-establishing step of the method set forth below. In addition, the polymeric material can have mechanical properties at a thickness of the sheath 154 sufficient to resist collapse of the sheath 154 into the needle slot 160 of the needle shaft 152 when the aspirating blood, notably, while also facilitating the cutting of the sheath 154 off the needle shaft 152 in accordance with the introducer needle-withdrawing step of the method set forth below. Such a polymeric material can include, but is not limited to, polyethylene, polypropylene, or polytetrafluoroethylene.

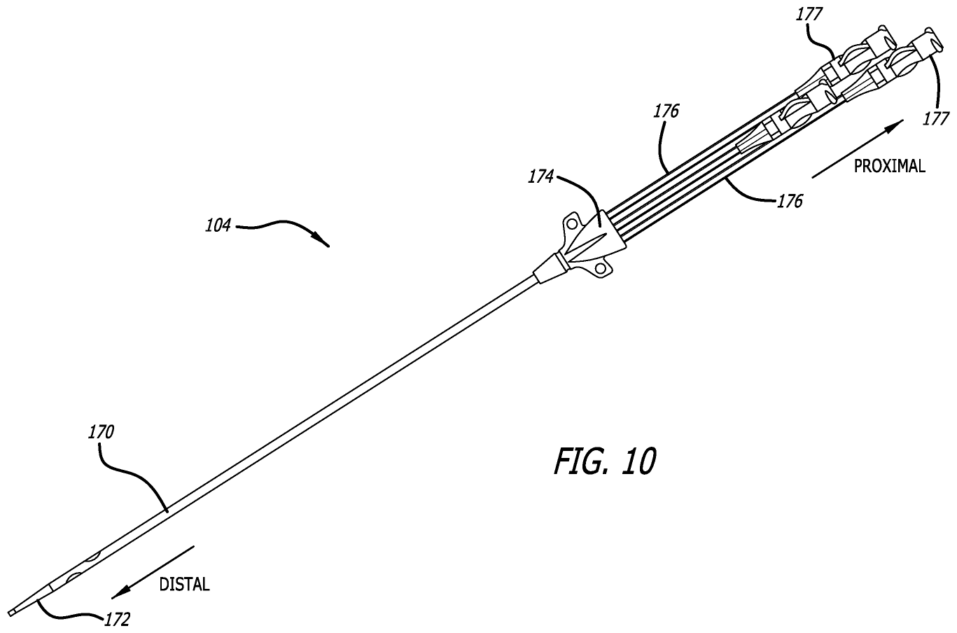

FIG. 10 illustrates the RICC 104 in accordance with some embodiments.

As shown, the RICC 104 can include a catheter tube 170 having a single-piece catheter tip 172 coupled thereto, a catheter hub 174, and one or more extension legs 176. The catheter tip 172 can be coupled to a distal end portion of the catheter tube 170, and the catheter hub 174 can be coupled to a proximal end portion of the catheter tube 170. Each extension leg of the one-or-more extension legs 176 can be coupled to the catheter hub 174 by a distal end portion thereof. In addition, one or more extension-leg connectors 177 (e.g., Luer connectors) can be coupled to the one-or-more extension legs 176 for connecting one or more other medical devices, respectively.

The RICC 104 can be a monoluminal RICC or a multiluminal RICC (e.g., a diluminal RICC, a triluminal RICC, a tetraluminal RICC, a pentaluminal RICC, a hexaluminal RICC, etc.). For example, the RICC 104 can be a triluminal RICC including a set of three lumens as shown in FIGS. 1 and 10. (See also FIG. 15.) The set of three lumens can include a primary lumen 178, a secondary lumen 180, and a tertiary lumen 182. Each lumen of the primary lumen 178, the secondary lumen 180, and the tertiary lumen 182 can be formed of at least three fluidly connected luminal portions, namely those of a catheter-tube lumen, a catheter-hub lumen, and an extension-leg lumen of three catheter-tube lumens, three catheter-hub lumens, and three extension-leg lumens in the triluminal RICC. For instance, the primary lumen 178 of the RICC 104 can be formed of a primary catheter-tube lumen 184, a primary catheter-hub lumen, and a primary extension-leg lumen; however, the primary lumen 178 of the RICC 104 can further include a fluidly connected catheter-tip lumen 186 of the catheter tip 172 as well. Further, the secondary lumen 180 of the RICC 104 can be formed of a secondary catheter-tube lumen 188, a secondary catheter-hub lumen, and a secondary extension-leg lumen. Lastly, the tertiary lumen 182 of the RICC 104 can be formed of a tertiary catheter-tube lumen 190, a tertiary catheter-hub lumen, and a tertiary extension-leg lumen. The primary lumen 178 can have a primary-lumen aperture 192 in a distal end of the catheter tip 172, which corresponds to a distal end of the RICC 104. The secondary lumen 180 can have a secondary-lumen aperture 194 in a side of the distal portion of the catheter tube 170. The tertiary lumen 182 can have a tertiary-lumen aperture 196 in the side of the distal portion of the catheter tube 170 proximal of the secondary-lumen aperture 194. Like the secondary and tertiary lumens 180 and 182, each additional lumen to the foregoing lumens can have a corresponding lumen aperture in the side of the distal portion of the catheter tube 170 proximal of its predecessor.

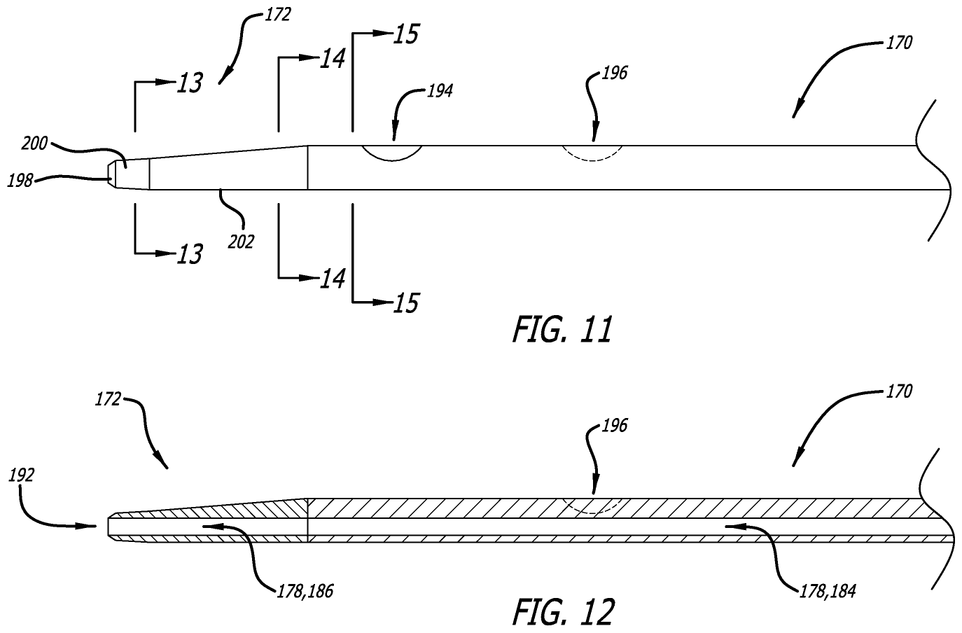

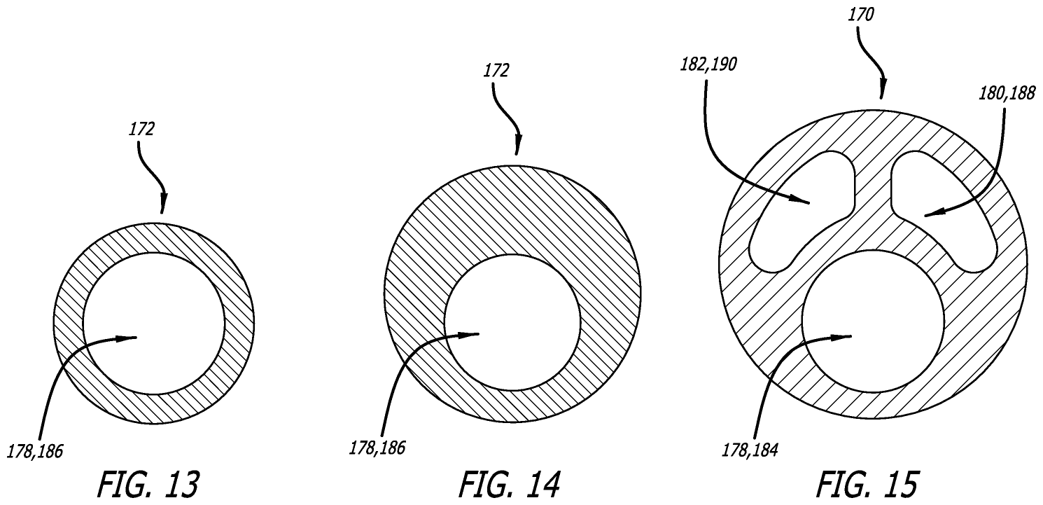

FIG. 11 illustrates the distal end portion of the catheter tube 170 and the catheter tip 172 coupled thereto in accordance with some embodiments. FIG. 12 illustrates a longitudinal cross section of the catheter tip 172 and the distal end portion of the catheter tube 170 in accordance with some embodiments, while FIGS. 13-15 illustrate various transverse cross section of the catheter tip 172 and the distal end portion of the catheter tube 170 in accordance with some embodiments.

As shown, the catheter tube 170 can include one or more catheter-tube lumens such as the three catheter-tube lumens set forth above. Indeed, when the RICC 104 is a triluminal RICC, the catheter tube 170 can include a catheter-tube portion of the primary lumen 178 of the RICC 104, a catheter-tube portion of the secondary lumen 180 of the RICC 104, and a catheter-tube portion of the tertiary lumen 182 of the RICC 104. Before forming the catheter tip 172 coupled to the catheter tube 170, each catheter-tube lumen of the foregoing three catheter-tube lumens can extend completely through the catheter tube 170. Subsequent to forming the catheter tip 172, however, only the primary catheter-tube lumen 184 typically extends from a proximal end of the catheter tube 170 to a distal end of the catheter tube 170. Indeed, the primary catheter-tube lumen 184 typically extends through both the proximal and distal ends of the catheter tube 170 and continues to extend through the catheter-tip lumen 186 of the catheter tip 172. In contrast, the secondary and tertiary catheter-tube lumens 188 and 190 typically terminate in the distal end portion of the catheter tube 170 by at least some infill of a melted polymeric material of the first polymeric material used to form the catheter tip 172.

The catheter tube 170 can be formed of a second, softer polymeric material than the first polymeric material of the catheter tip 172 set forth below. In other words, the second polymeric material can have a second durometer less than the first durometer of the first polymeric material. Such a second polymeric material can include, but is not limited to, a polyvinyl chloride, a polyethylene, a polyurethane, or a silicone having the second durometer less than the first durometer of the first polymeric material. For example, if the catheter tip 172 is formed of a polyurethane as set forth below, the catheter tube 170 can be formed of a different polyurethane (e.g., a same or different diisocyanate or triisocyanate reacted with a different diol or triol, a different diisocyanate or triisocyanate reacted with a same or different diol or triol, a same diisocyanate or triisocyanate reacted with a same diol or triol under different conditions or with different additives, etc.) having a second durometer less than a first durometer of the polyurethane of the catheter tip 172. Notably, polyurethanes can be advantageous for the catheter tube 170 in that polyurethanes are relatively stiff at room-temperature but become more pliable in vivo at body temperature, which reduces irritation to blood-vessel walls as well as phlebitis. Polyurethanes are also advantageous in that they can be less thrombogenic than some other polymers. Whether or not the second polymeric material is a polyurethane, the second polymeric material can be configured to be sufficiently stiff ex vivo to impart a column strength to the catheter tube 170 that prevents the catheter tube 170 from collapsing, buckling, or otherwise appreciably deforming when the RICC 104 is advanced into a blood-vessel lumen and placed therein.

As shown, the catheter tip 172 can have a first section 198, a second section 200, and a third section 202. The second section 200 of the catheter tip 172 can have a length l2 longer than a length l1 of the first section 198 of the catheter tip 172, and the third section 202 of the catheter tip 172 can have a length l3 longer than the length l2 of the second section 200 of the catheter tip 172.

The first section 198 of the catheter tip 172 can have a uniform taper having a constant first taper angle over an outer diameter thereof configured for immediately dilating tissue around a needle tract formed with the introducer needle 106 as the RICC 104 is advanced into a blood-vessel lumen of a patient. Like that set forth below with respect to the second section 200 of the catheter tip 172, the uniform taper can be visualized by successive transverse cross sections of the first section 198 of the catheter tip 172, which cross sections approximate larger concentric annuli in the proximal direction along the first section 198 of the catheter tip 172 and smaller concentric annuli in the distal direction along the first section 198 of the catheter tip 172. The uniform taper of the first section 198 of the catheter tip 172 can dilate the tissue around the needle tract from a size commensurate with an outer diameter of the needle shaft 152 of the introducer needle 106 to a size commensurate with the outer diameter of the second section 200 of the catheter tip 172, at its least. Being that the uniform taper of the first section 198 of the catheter tip 172 is not blunt like a catheter tip of a typical CVC, the first section 198 of the catheter tip 172 can reduce the force needed to insert the RICC 104 into the needle tract over that of the typical CVC.

The second section 200 of the catheter tip 172 can have a uniform taper having a constant second taper angle over an outer diameter thereof. Alternatively, the outer diameter of the second section 200 of the catheter tip 172 can be consistent along the second section 200 without the uniform taper. When present, the uniform taper of the second section 200 of the catheter tip 172 can be visualized by successive transverse cross sections of the second section 200 of the catheter tip 172, which cross sections approximate larger concentric annuli in the proximal direction along the second section 200 of the catheter tip 172 and smaller concentric annuli in the distal direction along the section of the catheter tip 172. One such transverse cross section is shown in FIG. 13. The uniform taper can be configured for immediately further dilating the tissue around the needle tract as the RICC 104 is advanced into the blood-vessel lumen of the patient. The uniform taper of the second section 200 of the catheter tip 172 can dilate the tissue around the needle tract from a size commensurate with the outer diameter of the first section 198 of the catheter tip 172, at its greatest, to a size commensurate with the outer diameter of the third section 202 of the catheter tip 172, at its least. Further being that the uniform taper of the second section 200 of the catheter tip 172 is not blunt like the catheter tip of the typical CVC, the second section 200 of the catheter tip 172 can further reduce the force needed to insert the RICC 104 into the needle tract over that of the typical CVC.

The third section 202 can have a non-uniform taper having a variable third taper angle over an outer diameter thereof configured for immediately further dilating the tissue around the needle tract as the RICC 104 is advanced into the blood-vessel lumen of the patient. The uniform taper of the third section 202 of the catheter tip 172 can be visualized by successive transverse cross sections of the third section 202 of the catheter tip 172, which cross sections approximate larger eccentric annuli in the proximal direction along the third section 202 of the catheter tip 172 and smaller eccentric annuli in the distal direction along the third section 202 of the catheter tip 172. One such transverse cross section is shown in FIG. 14. The non-uniform taper of the third section 202 of the catheter tip 172 can dilate the tissue around the needle tract from the size commensurate with the outer diameter of the second section 200 of the catheter tip 172, at its greatest, to a size commensurate with an outer diameter of the catheter tube 170. Further being that the non-uniform taper of the third section 202 of the catheter tip 172 is not blunt like the catheter tip of the typical CVC, the third section 202 of the catheter tip 172 can further reduce the force needed to insert the RICC 104 into the needle tract over that of the typical CVC. Indeed, the third section 202 of the catheter tip 172 is configured to integrate into the catheter tip 172 dilation normally done by a dilator in the Seldinger technique, thereby reducing the force needed to insert the RICC 104 into the needle tract and eliminating a separate dilation step.

The uniform taper of the first section 198 of the catheter tip 172 can have the constant first taper angle, the uniform taper of the second section 200 of the catheter tip 172 can, when present, have the constant second taper angle, and the non-uniform taper of the third section 202 of the catheter tip 172 can have the variable third taper angle. As shown by FIG. 11, the first taper angle can be greater than the third taper angle at a greatest taper angle of the third taper angle. That, and the third taper angle can be greater than the second taper angle at the greatest taper angle of the third taper angle. Notably, the third taper angle being associated with the non-uniform taper of the third section 202 of the catheter tip 172 can vary up from about 0° in accordance with the bottom edge of the longitudinal cross section of the third section 202 shown in FIG. 12 to the greatest taper angle in accordance with the bottom edge of the longitudinal cross section of the third section 202 shown in FIG. 12.

The catheter tip 172 can be formed of a first, harder polymeric material than the second polymeric material of the catheter tube 170 set forth above. In other words, the first polymeric material can have a first durometer greater than the second durometer of the second polymeric material. The first durometer can be a Shore A durometer of about 70-100, including a Shore A durometer of about 80-100, such as a Shore A durometer of about 90-100, for example, a Shore durometer of about 90-95. Such a first polymeric material can include, but is not limited to, a polytetrafluoroethylene, a polypropylene, or a polyurethane having the first durometer greater than the second durometer of the second polymeric material. For example, the catheter tip 172 can be formed of a polyurethane having the first durometer. Like that set forth above with respect to the catheter tube 170, polyurethanes can also be advantageous for the catheter tip 172. Whether or not the first polymeric material is a polyurethane, the first polymeric material can be configured to be sufficiently pliable in vivo at body temperature to prevent trauma to a blood-vessel lumen of a patient when the RICC 104 is advanced into the blood-vessel lumen per the method of placing the RICC 104 set forth below. However, the first polymeric material can be configured to soften less than the second polymeric material in vivo at body temperature, in a presence of moisture, or both. Indeed, the first polymeric material can be configured to remain sufficiently stiff to prevent the catheter tip 172 from collapsing, buckling, or otherwise appreciably deforming when the RICC 104 is advanced into the blood-vessel lumen of the patient. Additionally or alternatively, the first polymeric material can be configured to remain sufficiently stiff to prevent the catheter-tip lumen 186 of the catheter tip 172 from collapsing when aspirating through the catheter tip 172.

It should be understood that the first durometer of the first polymeric material and the second durometer of the second polymeric material can be on different hardness scales (e.g., Type A or Type D). With this understanding, the second durometer of the second polymeric material might not be numerically less than the first durometer of the first polymeric material when the second durometer is less than the first durometer. Indeed, the hardness of the second polymeric material can still be less than the hardness of the first polymeric material as the different hardness scales—each of which ranges from 0 to 100—are designed for characterizing different materials in groups of the materials having a like hardness.

The catheter hub 174 can include one or more catheter-hub lumens corresponding in number to the one-or-more catheter-tube lumens such as the three catheter-hub lumens set forth above, which correspond in number to the three catheter-tube lumens also set forth above. When the RICC 104 is a triluminal RICC, the catheter hub 174 can include a catheter-hub portion of the primary lumen 178 of the RICC 104, a catheter-hub portion of the secondary lumen 180 of the RICC 104, and a catheter-hub portion of the tertiary lumen 182 of the RICC 104. The one-or-more catheter-hub lumens can extend through an entirety of the catheter hub 174 from a proximal end of the catheter hub 174 to a distal end of the catheter hub 174.

The one-or-more extension legs 176 can respectively include one or more extension-leg lumens, which, in turn, correspond in number to the one-or-more catheter-hub lumens such as the three extension-leg lumens set forth above, which correspond in number to the three catheter-hub lumens also set forth above. When the RICC 104 is a triluminal RICC, the one-or-more extension legs 176 can include a primary extension leg including an extension-leg portion of the primary lumen 178 of the RICC 104, a secondary extension leg including an extension-leg portion of the secondary lumen 180 of the RICC 104, and a tertiary extension leg including an extension-leg portion of the tertiary lumen 182 of the RICC 104. Each extension-leg lumen of the one-or-more extension-leg lumens can extend through an entirety of its corresponding extension leg from a proximal end of the extension leg to a distal end of the extension leg.

Notably, any component of the RICC 104 selected from the catheter tip 172, the catheter tube 170, the catheter hub 174, the one-or-more extension legs 176, and the one-or-more extension-leg connectors 177 can include an antimicrobial thereon or therein. In an example, the catheter tube 170 and the catheter tip 172 coupled thereto can include an antimicrobial coating on their abluminal surfaces. In another example, a pre-extrusion material of the catheter tube 170 such as the second polymeric material can include the antimicrobial admixed therein such that the antimicrobial is incorporated into the catheter tube 170 when extruded, the antimicrobial protecting both an abluminal surface of the catheter tube 170 and a luminal surface of the catheter tube 170 from microbial contamination. Additionally or alternatively, a polymeric plug of the first polymeric material tube can include the antimicrobial admixed therein such that the antimicrobial is incorporated into the catheter tip 172 when the catheter tip 172 is formed by, for example, inserting the polymeric plug into either a secondary or tertiary catheter-tube lumen of a cut end of catheter-tube tubing and forming the catheter tip 172 with molten polymeric material of the polymeric plug over a mandrel in a cavity of a radiofrequency (“RF”)-welding die. The antimicrobial in such a catheter tip 172 can protect both an abluminal surface of the catheter tip 172 and a luminal surface of the catheter tip 172 from microbial contamination.

As shown, the RICC insertion assembly 100 or the introducer-needle subassembly 102 thereof can further include an access guidewire 204. The access guidewire 204 can include a proximal portion having a proximal end and a distal portion having a distal end. The distal portion of the access guidewire 204 can pass through the guidewire passageway 124 of the combination of coupler components selected from the coupler housing 114, the valve-module housing 118, and the elastomeric gasket 120, through the sheath opening 168 in the sheath 154, and into the needle lumen 164 of the introducer needle 106 such that the distal end of the access guidewire 204 is disposed just proximal of the needle tip 158 of the needle shaft 152 in the ready-to-deploy state of the RICC insertion assembly 100 or the introducer-needle subassembly 102. Again, the proximal end of the access guidewire 204 can be attached to the extension arm 110, and the proximal and distal ends of the access guidewire 204 can enforce the loop 112 in the access guidewire 204 in at least the ready-to-deploy state of the RICC insertion assembly 100, which loop 112 the RICC 104 can be disposed over, thereby keeping the RICC insertion assembly 100 in a relatively compact form.

The access guidewire 204 can include a guidewire tip 206 in the distal portion of the access guidewire 204, which adopts a ‘J’ shape configured to prevent puncturing a back wall of a blood vessel. Such a guidewire tip 206 can assume a straightened state in at least the ready-to-deploy state of the RICC insertion assembly 100 and a curved state when the guidewire tip 206 is advanced beyond the needle tip 158 (e.g., advanced into a blood-vessel lumen) in a deployed state of the RICC insertion assembly 100.

As shown, the RICC insertion assembly 100 or the introducer-needle subassembly 102 can further include a syringe 208. The syringe 208 can be fluidly coupled to the needle hub 156 of the introducer needle 106 in at least the ready-to-deploy state of the RICC insertion assembly 100. As set forth above, the sheath 154 can seal the needle slot 160 of the needle shaft 152 thereunder. In particular, the sheath 154 can seal the needle slot 160 outside of the valve module 116. The valve module 116, in turn, can seal over the sheath opening 168 of the sheath 154. The valve module 116 can also seal around the access guidewire 204. Such seals enable the syringe 208 to aspirate blood, for example, to confirm blood-vessel access subsequent to establishing a needle tract in accordance with the needle tract-establishing step of the method set forth below.

### Methods

Methods can include methods of the RICC insertion assembly 100 or the introducer-needle subassembly 102. For example, a method of at least the introducer-needle subassembly 102 can include one or more steps selected from a needle tract-establishing step, an access guidewire-advancing step, an introducer needle-withdrawing step, and a needle tip-capturing step.

The needle tract-establishing step can include establishing a needle tract from a skin surface to a blood vessel of a patient with the introducer needle 106 of the RICC insertion assembly 100 or the introducer-needle subassembly 102. As set forth above, the introducer needle 106 can include the needle hub 156 over the proximal portion of the composite shaft 150. And the composite shaft 150 can include the needle shaft 152 and the sheath 154 over the needle shaft 152 sealing the needle slot 160 thereunder but for the sheath opening 168 in the sheath 154 to the needle slot 160.

The access guidewire-advancing step can include advancing the distal end of the access guidewire 204 from just proximal of the needle tip 158 of the needle shaft 152 into the blood vessel.

The introducer needle-withdrawing step can include withdrawing the introducer needle 106 in the proximal direction from the needle tract leaving the access guidewire 204 in place in the blood vessel. The introducer needle-withdrawing step can also include further withdrawing the introducer needle 106 in the proximal direction from the coupler 108 coupling the introducer needle 106 and the access guidewire 204 together. As set forth above, the coupler 108 can includes the coupler housing 114 and the valve module 116 disposed in the coupler housing 114.

The needle tip-capturing step can include capturing the needle tip 158 of the needle shaft 152 within the valve module 116 by way of the NSI protection mechanism 130.

As set forth above, the NSI protection mechanism 130 can include the tether 134 having the proximal portion coupled to the needle hub 156 and the distal portion coupled to the valve-module housing 118. The tether 134 can have the fixed tether length configured to prevent the needle tip 158 of the needle shaft 152 from escaping the valve module 116 during the further withdrawing of the introducer needle 106 from the coupler 108 in the introducer needle-withdrawing step or thereafter. In addition, the tether 134 can include the pleats 136 and the pleat through holes 138 through the pleats 136. The composite shaft 150 can be disposed in the pleat through holes 138 such that the pleats 136 unfold over the composite shaft 150 during the further withdrawing of the introducer needle 106 from the coupler 108 in the introducer needle-withdrawing step.

As further set forth above, the NSI protection mechanism 130 can include the spring 128 loaded in the spring cavity 132 along the portion of the introducer-needle passageway 122 defined by the valve-module housing 118 of the valve module 116. The spring 128 can unload into the introducer-needle passageway 122 during the further withdrawing of the introducer needle 106 from the coupler 108 in the introducer needle-withdrawing step at the time when the needle tip 158 of the needle shaft 152 passes the spring 128. Again, the spring 128 can be a flat spring including the end tab 140 configured to extend across the introducer-needle passageway 122 into the tab receiver 142 of the valve-module housing 118 opposite the spring cavity 132 at the time the needle tip 158 of the needle shaft 152 passes the spring 128. This prevents the needle tip 158 of the needle shaft 152 from escaping the valve module 116 if the introducer needle 106 is advanced into the coupler 108 in a distal direction. Notably, the end tab 140 of the spring 128 can provide the primary tip shield, and the fulcrum 144 of the spring 128 can provide the secondary tip shield if the needle tip 158 of the needle shaft 152 is forcibly advanced into the coupler 108 in the distal direction past the end tab 140. The fulcrum 144 of the spring 128 can be between the free arm 146 of the spring 128 including the end tab 140 and the fixed arm 148 of the spring 128 fixed to the valve-module housing 118 in the spring cavity 132.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

1. A rapidly insertable central catheter (“RICC”) insertion assembly, comprising:
a RICC;
an introducer needle including a needle hub over a proximal portion of a composite shaft, the composite shaft including a needle shaft having a longitudinal needle slot and a sheath over the needle shaft sealing the needle slot thereunder but for a sheath opening in the sheath to the needle slot;
a coupler coupling the RICC and the introducer needle together, the coupler including:
a coupler housing; and
a valve module disposed in the coupler housing, the composite shaft passing through an introducer-needle passageway defined by a valve-module housing of the valve module such that the valve module forms a seal over the sheath opening in a ready-to-deploy state of the RICC insertion assembly, the valve module including a needle-stick-injury (“NSI”) protection mechanism configured to capture a distal needle tip of the needle shaft within the valve module when the introducer needle is withdrawn from the coupler in a proximal direction.

2. The RICC insertion assembly of claim 1, wherein the NSI protection mechanism includes a tether having a proximal portion coupled to the needle hub and a distal portion coupled to the valve-module housing.

3. The RICC insertion assembly of claim 2, wherein the tether has a fixed tether length configured to prevent the needle tip of the needle shaft from escaping the valve module when the introducer needle is withdrawn from the coupler in the proximal direction or thereafter.

4. The RICC insertion assembly of claim 2, wherein the tether includes a plurality of pleats and a plurality of pleat through holes through the pleats, the composite shaft disposed in the pleat through holes such that the pleats unfold over the composite shaft when the introducer needle is withdrawn from the coupler in a proximal direction.

5. The RICC insertion assembly of claim 1, wherein the NSI protection mechanism includes a spring loaded in a spring cavity along a portion of the introducer-needle passageway, the spring configured to unload into the introducer-needle passageway when the introducer needle is withdrawn from the coupler in the proximal direction and the needle tip of the needle shaft passes the spring.

6. The RICC insertion assembly of claim 5, wherein the spring is a flat spring including an end tab configured to extend across the introducer-needle passageway into a tab receiver of the valve-module housing opposite the spring cavity at the time the needle tip of the needle shaft passes the spring, thereby preventing the needle tip of the needle shaft from escaping the valve module if the introducer needle is advanced into the coupler in a distal direction.

7. The RICC insertion assembly of claim 6, wherein the end tab of the spring provides a primary tip shield, and a fulcrum of the spring provides a secondary tip shield if the needle tip of the needle shaft is forcibly advanced into the coupler in the distal direction past the end tab.

8. The RICC insertion assembly of claim 7, wherein the fulcrum of the spring is between a free arm of the spring including the end tab and a fixed arm of the spring fixed to the valve-module housing in the spring cavity.

9. The RICC insertion assembly of claim 7, further comprising:
an access guidewire including a distal portion having a distal end, the distal portion of the access guidewire passing through a guidewire passageway defined by the valve-module housing of the valve module, a continuation of the guidewire passageway defined by an elastomeric gasket of the valve module, the sheath opening in the sheath, and an introducer-needle lumen of the introducer needle such that the distal end of the access guidewire is disposed just proximal of the needle tip of the needle shaft in the ready-to-deploy state of the RICC insertion assembly.

10. The RICC insertion assembly of claim 9, wherein the valve module includes a blade extending into the sheath opening of the sheath from the valve-module housing to which the blade is fixed, the blade configured to cut the sheath along the needle slot when the introducer needle is withdrawn from the coupler in a proximal direction, thereby allowing the access guidewire to escape from the introducer-needle lumen of the introducer needle.

11. The RICC insertion assembly of claim 1, wherein the RICC includes:
a catheter tube including a catheter-tube portion of a primary lumen of the RICC;
a single-piece catheter tip coupled to a distal end portion of the catheter tube;
a catheter hub coupled to a proximal portion of the catheter tube; and
one or more extension legs, each extension leg of the one-or-more extension legs coupled to the catheter hub by a distal portion thereof.

12. The RICC insertion assembly of claim 11, wherein the catheter tip is of a first polymeric material and the catheter tube is of a second, softer polymeric material.

13. The RICC insertion assembly of claim 12, wherein the first polymeric material is sufficiently stiff to prevent the catheter tip from collapsing, buckling, or otherwise appreciably deforming when a distal portion of the RICC is advanced into a blood-vessel lumen of a patient, the first polymeric material also being sufficiently pliable to prevent trauma to the blood-vessel lumen when the distal portion of the RICC is advanced farther into the blood-vessel lumen.

14. The RICC insertion assembly of claim 11, wherein the RICC includes a set of three lumens including the primary lumen, a secondary lumen, and a tertiary lumen formed of fluidly connected portions of at least three catheter-tube lumens, three catheter-hub lumens, and three extension-leg lumens, the secondary and tertiary lumens terminating in the distal end portion of the catheter tube by at least some infill of melted polymeric material of the first polymeric material.

15. The RICC insertion assembly of claim 14, wherein the primary lumen has a primary-lumen aperture in a distal end of the catheter tip, the secondary lumen has a secondary-lumen aperture in a side of the distal portion of the catheter tube, and the tertiary lumen has a tertiary-lumen aperture in the side of the distal end portion of the catheter tube but proximal of the secondary-lumen aperture.

16. The RICC insertion assembly of claim 1, further comprising:
a syringe fluidly coupled to the needle hub of the introducer needle in at least the ready-to-deploy state of the RICC insertion assembly.

17. An introducer-needle subassembly, comprising:
an introducer needle including a needle hub over a proximal portion of a composite shaft, the composite shaft including a needle shaft having a longitudinal needle slot and a sheath over the needle shaft sealing the needle slot thereunder but for a sheath opening in the sheath to the needle slot;
a coupler coupled to the introducer needle, the coupler including:
a coupler housing; and
a valve module disposed in the coupler housing, the composite shaft passing through an introducer-needle passageway defined by a valve-module housing of the valve module such that the valve module forms a seal over the sheath opening in a ready-to-deploy state of the introducer-needle subassembly, the valve module including a needle-stick-injury (“NSI”) protection mechanism configured to capture a distal needle tip of the needle shaft within the valve module when the introducer needle is withdrawn from the coupler in a proximal direction.

18. The introducer-needle subassembly of claim 17, wherein the NSI protection mechanism includes a tether having a proximal portion coupled to the needle hub and a distal portion coupled to the valve-module housing.

19. The introducer-needle subassembly of claim 18, wherein the tether has a fixed tether length configured to prevent the needle tip of the needle shaft from escaping the valve module when the introducer needle is withdrawn from the coupler in the proximal direction or thereafter.

20. The introducer-needle subassembly of claim 18, wherein the tether includes a plurality of pleats and a plurality of pleat through holes through the pleats, the composite shaft disposed in the pleat through holes such that the pleats unfold over the composite shaft when the introducer needle is withdrawn from the coupler in a proximal direction.

21. The introducer-needle subassembly of claim 17, wherein the NSI protection mechanism includes a spring loaded in a spring cavity along a portion of the introducer-needle passageway, the spring configured to unload into the introducer-needle passageway when the introducer needle is withdrawn from the coupler in the proximal direction and the needle tip of the needle shaft passes the spring.

22. The introducer-needle subassembly of claim 21, wherein the spring is a flat spring including an end tab configured to extend across the introducer-needle passageway into a tab receiver of the valve-module housing opposite the spring cavity at the time the needle tip of the needle shaft passes the spring, thereby preventing the needle tip of the needle shaft from escaping the valve module if the introducer needle is advanced into the coupler in a distal direction.

23. The introducer-needle subassembly of claim 22, wherein the end tab of the spring provides a primary tip shield, and a fulcrum of the spring provides a secondary tip shield if the needle tip of the needle shaft is forcibly advanced into the coupler in the distal direction past the end tab.

24. The introducer-needle subassembly of claim 23, wherein the fulcrum of the spring is between a free arm of the spring including the end tab and a fixed arm of the spring fixed to the valve-module housing in the spring cavity.

25-32. (canceled)
